(19) United States
(12) Patent Application Publication
(10) Pub. No.: US 2021/0198695 A1
(43) Pub. Date: Jul. 1, 2021

(54) PRODUCTION METHODS FOR VIRAL VECTORS

(71) Applicant: Spacecraft Seven, LLC, Cranbury, NJ (US)

(72) Inventors: David RICKS, Cranbury, NJ (US); Brian BEARD, Cranbury, NJ (US); Kenneth LAW, Cranbury, NJ (US); Raj PRABHAKAR, Cranbury, NJ (US); Kinnari PATEL, Cranbury, NJ (US)

(21) Appl. No.: 17/268,409

(22) PCT Filed: Aug. 16, 2019
(86) PCT No.: PCT/US2019/046890

§ 371 Date: Feb. 12, 2021

Related U.S. Application Data
(60) Provisional application No. 62/765,112, filed on Aug. 16, 2018.

(51) Int. Cl. C12N 15/86; A61K 35/76

(52) U.S. Cl. CPC C12N 15/86; A61K 35/76; C12N 2740/16032; C12N 2740/16052; C12N 2740/16043

(57) ABSTRACT

The present disclosure provides methods for manufacturing a recombinant lentiviral vectors in an adherent bioreactor, for example, by calcium-phosphate transfection of cells grown in adherent mode on low-compaction macrocarriers in an iCELLis® bioreactor system.

### RELATED APPLICATIONS

This application claims priority to U.S. Provisional Patent Appl. No. 62/765,112, filed Aug. 16, 2018, which is incorporated herein by reference in its entirety.

### FIELD OF THE INVENTION

The present invention relates generally to methods for manufacturing recombinant lentiviral vectors.

### BACKGROUND OF THE INVENTION

Successful manufacturing of recombinant lentiviral vectors in an adherent bioreactor can be challenging because yields are dependent on numerous process parameters and few reports of optimized process parameters are available in the public domain. It has recently been reported that use of polyethylenimine (PEI) as a transfection reagent achieves superior yields to calcium phosphate (CaPho) precipitation-based transfection. Valkama et al. Gene Therapy (2018) 254, 39-46 (2018). Despite such reports, the present inventors have developed optimized methods that rely of CaPho transfection.

### BRIEF DESCRIPTION OF THE DRAWINGS

FIG. 1 shows a process diagram depicting an illustrative non-limiting embodiment of the methods of the disclosure.

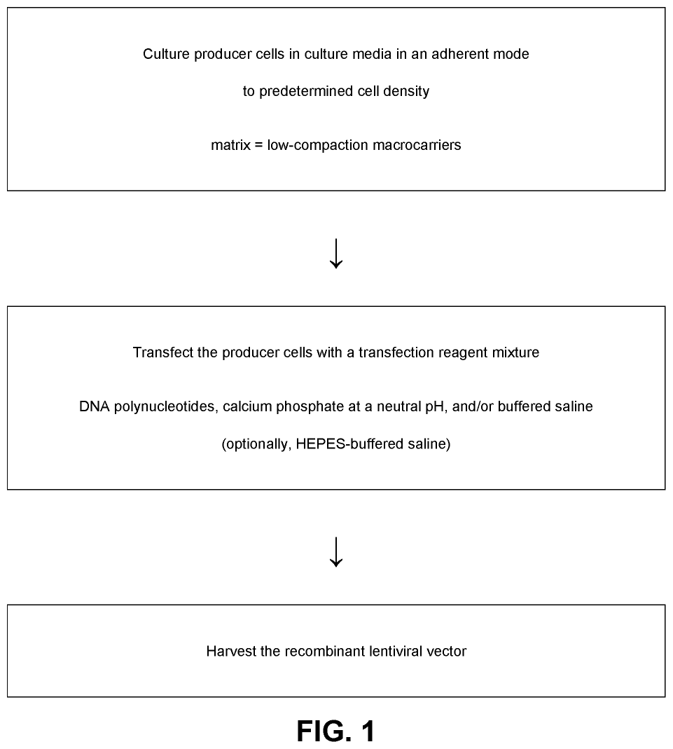

FIG. 2 shows the iCELLis® bioreactor bench (Nano) and manufacturing (500) scales. LC=low compaction; HC=high compaction. Source: https://biotech.pall.com/

### SUMMARY OF THE INVENTION

The present disclosure provides a method of manufacturing a recombinant lentiviral vector, comprising culturing producer cells in culture media in an adherent mode on a matrix, wherein the matrix comprises low-compaction macrocarriers, in an adherent bioreactor having a bed height and a reactor volume until the producer cells achieve a predetermined cell density; transfecting the producer cells with a transfection reagent mixture, wherein the transfection reagent mixture comprises one or more DNA polynucleotides, calcium phosphate at a neutral pH, and buffered saline (e.g., HEPES-buffered saline); and harvesting the recombinant lentiviral vector, thereby generating harvested material. In some embodiments, the method comprises processing the harvested material using a semi-closed or closed system, thereby generating purified material.

Other features and advantages of the invention will be apparent from and encompassed by the following detailed description and claims.

### DETAILED DESCRIPTION

The present disclosure provides inter alia a method of manufacturing a recombinant lentiviral vector, comprising culturing producer cells in culture media in an adherent mode on a matrix, wherein the matrix comprises low-compaction macrocarriers, in an adherent bioreactor having a bed height and a reactor volume, until the producer cells achieve a predetermined cell density; transfecting the producer cells with a transfection reagent mixture, wherein the transfection reagent mixture comprises one or more DNA polynucleotides, calcium phosphate at a neutral pH, and buffered saline (e.g., HEPES-buffered saline); and harvesting the recombinant lentiviral vector, thereby generating harvested material. The present disclosure also provides recombinant lentiviral vectors produced by the methods disclosed herein, as well as pharmaceutical compositions and uses of the lentiviral vectors and pharmaceutical compositions.

The compositions and methods of the present disclosure are particularly suitable for gene therapy applications, including the treatment of monogenic diseases and disorders. Factors that have limited gene therapy success, including low yields of recombinant lentiviral vectors in manufacturing, are solved by the compositions and methods provided herein.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Various embodiments contemplated herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Oligonucleotide Synthesis (M. J. Gait, ed., 1984); Animal Cell Culture (R. I. Freshney, ed., 1987); “Methods in Enzymology” (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); PCR: The Polymerase Chain Reaction (Mullis et al., eds., 1994), Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology J. E. Coligan, A. M. Kruisbeek, D. H. Margulies, E. M. Shevach and W. Strober, eds., 1991); Annual Review of Immunology; as well as monographs in journals such as Advances in Immunology, each of which is expressly incorporated by reference herein.

In the following description, certain specific details are set forth in order to provide a thorough understanding of various illustrative embodiments of the invention contemplated herein. However, one skilled in the art will understand that particular illustrative embodiments may be practiced without these details. In addition, it should be understood that the individual vectors, or groups of vectors, derived from the various combinations of the structures and substituents described herein, are disclosed by the present application to the same extent as if each vector or group of vectors was set forth individually. Thus, selection of particular vector structures or particular substituents is within the scope of the present disclosure.

### Definitions

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

“Transfection” refer to the process of introducing naked DNA into cells by non-viral methods.

“Infection” refers to the process of introducing foreign DNA into cells using a viral vector.

“Transduction” refers to the introduction of foreign DNA into a cell using a viral vector.

“Vector copy number” or “VCN” refers to the number of copies of vector in a sample divided by the number of cells. Generally, the number of copies of vector is determined by quantitative polymerase chain reaction (qPCR) using a probe against the Psi sequence of the integrated provirus, and the number of cells is determined by qPCR using a probe against a human housekeeping gene for which there will be two copies per cell (one per chromosome).

“Transduction efficiency” refers to the percentage of cells transduced with at least one provirus copy. For example if 1×10⁶ cells are exposed to a virus and 0.5×10⁶ cells are determined to have a least one copy of a virus in their genome, then the transduction efficiency is 50%. An illustrative method for determining transduction efficiency is flow cytometry.

As used herein, the term “retrovirus” or “retroviral” refers an RNA virus that reverse transcribes its genomic RNA into a linear double-stranded DNA copy and subsequently covalently integrates its genomic DNA into a host genome. Retrovirus vectors are a common tool for gene delivery (Miller, 2000, Nature. 357: 455-460). Once the virus is integrated into the host genome, it is referred to as a “provirus.” The provirus serves as a template for RNA polymerase II and directs the expression of RNA molecules encoded by the virus. Illustrative retroviruses (family Retroviridae) include, but are not limited to: (1) the genus gammaretrovirus, such as, e.g., Moloney murine leukemia virus (M-MuLV), Moloney murine sarcoma virus (MoMSV), murine mammary tumor virus (MuMTV), gibbon ape leukemia virus (GaLV), and feline leukemia virus (FLV), (2) the genus spumavirus, such as, e.g., simian foamy virus, and (3) the genus lentivirus, such as, e.g., human immunodeficiency virus-1 and simian immunodeficiency virus.

As used herein, the term “lentiviral” or “lentivirus” refers to a group (or genus) of complex retroviruses. Illustrative lentiviruses include, but are not limited to: human immunodeficiency virus (HIV), including HIV type, and HIV type 2; visna-maedi virus (VMV) virus; the caprine arthritis-encephalitis virus (CAEV); equine infectious anemia virus (EIAV); feline immunodeficiency virus (Hy); bovine immune deficiency virus (BIV); and simian immunodeficiency virus (SIV). In one embodiment, HIV-based vector backbones (i.e., HIV cis-acting sequence elements) are utilized.

Retroviral vectors, and in particular embodiments, lentiviral vectors, may be used in practicing the present invention. Accordingly, the term “retroviral vector,” as used herein is meant to include “lentiviral vector”; and the term “retrovirus” as used herein is meant to include “lentivirus.”

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication or reverse transcription in a cell, or may include sequences sufficient to allow integration into host cell DNA. Useful vectors include viral vectors. Useful viral vectors include, e.g., replication defective retroviruses and lentiviruses.

The term “viral vector” may refer either to a viral-based vector or vector particle capable of transferring a nucleic acid into a cell or to the transferred nucleic acid itself. Viral vectors contain structural and/or functional genetic elements that are primarily derived from a virus. The term “retroviral vector” refers to a viral vector containing structural and functional genetic elements, or portions thereof, that are primarily derived from a retrovirus.

The term “lentiviral vector” refers to a viral vector containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In one embodiment, a hybrid vector comprises retroviral, e.g., lentiviral, sequences for reverse transcription, replication, integration and/or packaging.

In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the invention and are present in DNA form in the DNA plasmids of the invention.

According to certain specific embodiments, most or all of the viral vector backbone sequences are derived from a lentivirus, e.g., HIV-1. However, it is to be understood that many different sources of lentiviral sequences can be used, and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid of the present invention.

As used herein, the terms “polynucleotide” or “nucleic acid” refers to DNA and RNA, e.g., genomic DNA (gDNA), complementary DNA (cDNA) or DNA. Polynucleotides include single and double stranded polynucleotides, either recombinant, synthetic, or isolated. In some embodiments, polynucleotide refers to messenger RNA (mRNA), RNA, genomic RNA (gRNA), plus strand RNA (RNA(+)), minus strand RNA (RNA(−)). As used here, the terms “polyribonucleotide” or “ribonucleic acid” also refer to messenger RNA (mRNA), RNA, genomic RNA (gRNA), plus strand RNA (RNA(+)), minus strand RNA (RNA(−)). Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, viral vector and transfer plasmid polynucleotide sequences and compositions comprising the same are contemplated. In particular embodiments, polynucleotides encoding one or more therapeutic polypeptides and/or other genes of interest are contemplated. In particular embodiments, polynucleotides encoding a therapeutic polypeptide including, but not limited to, RPK, ITGB2, FANCA, FANCC, FANCG, TCIRG1, CLCN7, TNFSF11, PLEKHM1, TNFRSF11A and OSTM1 genes. In particular embodiments, polynucleotides or regions thereof encoding a therapeutic polypeptide are codon-optimized.

By “enhance” or “promote,” or “increase” or “expand” refers generally to the ability of the compositions and/or methods contemplated herein to elicit, cause, or produce higher numbers of cells, higher numbers of transduced cells, or higher yield of viral compared to methods performed in an adherent bioreactor under control conditions. An “increased” or “enhanced” yield is typically a “statistically significant” amount, and may include an increase that is 1.1, 1.2, 1.5, 2, 3, 4, 5, 6, 7, 8, 9, 10, 15, 20, 30, 50, 100, 200 or more times (e.g., 500, 1000 times) (including all integers and decimal points in between and above 1, e.g., 1.5, 1.6, 1.7. 1.8, etc.) the yield of the adherent bioreactor under control conditions.

As used herein, “control conditions” refers to process conditions prior to optimization. Control conditions may refer, for example, to Example 1 (Ex. 1) in Table A, or equivalent conditions.

By “decrease” or “lower,” or “lessen,” or “reduce,” or “abate” refers generally to compositions or methods that result in comparably fewer total cells, fewer transduced cells, or lower yield compared to methods performed in an adherent bioreactor under control conditions. A “decrease” or “reduced” yield is typically a “statistically significant” amount, and may include an decrease that is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more percent (e.g., 40%, 50%, 60%) (including all integers and decimal points in between and above 1, e.g., 1.5, 1.6, 1.7. 1.8, etc.) decreased compared to the yield of the adherent bioreactor under control conditions.

As used herein, “CFC” refers to colony forming cells. The colony forming cell (CFC) assay is used to study the proliferation and differentiation pattern of hematopoietic progenitors by their ability to form colonies in a semisolid medium. The number and the morphology of the colonies formed by a fixed number of input cells provide preliminary information about the ability of progenitors to differentiate and proliferate. Exemplary assays are provided in Sarma et al. Colony forming cell (CFC) assay for human hematopoietic cells. J Vis Exp. 2010 Dec. 18; (46).

As used herein, “CFU” refers to colony forming units. CFU is understood to be synonymous with CFC, but is sometimes used in reference to the types of CFUs growing in semisolid media.

As used herein, “TU” refers to transducing units. TU/mL is a common measurement of the functional titer of a retroviral (lentiviral) preparation.

As used herein, “MOI” refers to multiplicity of infection.

The terms “administering” or “introducing”, as used herein, refer to delivery of a lentiviral vector, or of cells transduced with a lentiviral vector, to a subject

Typically, a cell is referred to as “transduced” when a viral vector or vector particle has introduced heterologous DNA (e.g., the vector) into the cell.

The term “host cell”, as used herein refers to a cell which has been transduced with a viral vector or vector particle. It will be appreciated that the term “host cell” refers to the original transduced cell and progeny thereof.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

### Methods of Manufacture

In an embodiment, the disclosure provides a method of manufacturing a recombinant lentiviral vector, wherein: producer cells are cultured in culture media in an adherent mode on a matrix, wherein the matrix comprises low-compaction macrocarriers, in an adherent bioreactor having a bed height and a reactor volume until the producer cells achieve a predetermined cell density; the producer cells are transfected with a transfection reagent mixture; and recombinant lentiviral vector is harvested from the transfected cell. In some embodiments, the transfection reagent mixture comprises one or more DNA polynucleotides, calcium phosphate at a neutral pH, and/or HEPES-buffered saline. The recombinant lentiviral vector may be referred to as “harvested material.”

In an embodiment, the transfecting step comprises adding to the adherent bioreactor about a 5% to about a 50% volume of the transfection reagent mixture for each 100% volume of transfection reagent mixture and culture media combined. In an embodiment, the transfecting step comprises adding to the adherent bioreactor about a 10% to about a 40% volume of the transfection reagent mixture for each 100% volume of transfection reagent mixture and culture media combined. In an embodiment, the transfecting step comprises adding to the adherent bioreactor about a 10%, 15%, 20%, 25%, 30%, 35%, or 40% volume of the transfection reagent mixture for each 100% volume of transfection reagent mixture and culture media combined. In an embodiment, the transfecting step comprises adding to the adherent bioreactor about 0.8, 0.9, 1.0, 1.1, or 1.2 volumes of the transfection reagent mixture for each 3 volumes of culture media. In an embodiment, the transfecting step comprises adding to the adherent bioreactor about 1 volume of the transfection reagent mixture for each 3 volumes of culture media.

In an embodiment, the method comprises, after the transfecting step, waiting for a time period of at least about 1, 2, 3, 5, 6, 7, 8, 9, 10, 11, 12, 13, or 14 hours or longer, e.g., 4-24 hours, 8-24 hours, 8-14 hours, 4-12 hours or 5-7 hours, before the harvesting step, e.g., a time sufficient to allow production of the viral vector.

In an embodiment, the method comprises, after the transfecting step, a step of recirculating the culture media through the matrix for at least about 1, 2, 3, 5, 6, 7, 8, 9, 10, 11, 12, 13, or 14 hours or longer while maintaining the pH at a fixed pH, such as about pH 6.7, 6.8, 6.9. 7.0, 7.1, 7.2, 7.3, or 7.4. In an embodiment, the method comprises, after the transfecting step, recirculating the culture media through the matrix for at least about 5-7 hours while maintaining the pH at about 7.2.

In an embodiment, the harvesting step comprises maintaining the pH of the culture media less than or slightly less than the pH of the culturing step, e.g. about pH in the range of about 6.0 to about 7.3. In an embodiment, the harvesting step comprises maintaining the pH of the culture media less than or slightly less than the pH of the culturing step, e.g. at pH 6.3, 6.4, 6.5, 6.6, 6.7, 6.8, 6.9. 7.0, 7.1, or 7.2. In an embodiment, the harvesting step comprises maintaining the pH of the culture media at less than about pH 7.0.

In an embodiment, the harvesting step comprises perfusing the matrix with at least about 4 reactor volumes of harvesting media for at least about 24, 48, 60, or 72 hours, or in the range of 24 to 72 hours. In an embodiment, the harvesting step comprises perfusing the matrix with about 4 reactor volumes of harvesting media for about 60 hours. In an embodiment, the harvesting step comprises perfusing the matrix with harvesting media for about 60 hours and collecting the media or portions thereof at regular time intervals, e.g., every 12, 24, 36, or 48 hours.

In an embodiment, the method comprises processing the harvested material using a semi-closed or closed system, thereby generating purified material.

In an embodiment, the processing step comprises one or more of ion exchange chromatography and size exclusion chromatography.

In an embodiment, the processing step comprises concentrating the recombinant lentiviral vector by centrifugation of the harvested material in one or more centrifugal concentrators. In an embodiment, the processing step comprises concentrating the recombinant lentiviral vector by tangential flow filtration.

In an embodiment, the method comprises assaying the purified material for an infectious titer of the recombinant lentiviral vector. In an embodiment, the recombinant lentiviral vector manufactured by the method exhibits viral transduction efficacy that is increased at least about 20% compared to recombinant lentiviral vector not so manufactured. In an embodiment, the recombinant lentiviral vector manufactured by the method exhibits viral transduction efficacy that is increased at least about 20% compared to recombinant lentiviral vector manufactured without optimization of process parameters as described herein, for example, bioreactors 1-4 of run 1 as described in Example 1.

Various other methodology for purifying lentiviral vectors can be used.

Bioreactors, Macrocarriers and Production Systems

Certain aspects of the present disclosure relate to methods for culturing a cell in a fixed bed. Illustrative cell culture devices are referenced in U.S. Pat. Nos. 8,597,939, 8,137,959, US PG Pub 2008/0248552, and WO2014093444 and are commercially available (e.g., the iCELLis® Bioreactors from Pall® Life Sciences, Port Washington, N.Y., such as the Nano and 500/100 bioreactors). The iCELLis® Bioreactors are designed to permit scaling of manufacturing conditions from the Nano bioreactor to larger bioreactors without appreciable changes in results. Thus, manufacturing conditions developed on the Nano bioreactor translate to any iCELLis® bioreactor. It is also possible to use other current or prospectively created fixed-bed bioreactors in the methods of the present disclosure. In some embodiments, the cell is cultured in a fixed-bed bioreactor. Fixed-bed bioreactors include a carrier in the form of a stationary packing material forming a fixed or packed bed for promoting cell adhesion and growth. The arrangement of the packing material of the fixed bed affects local fluid, heat, and mass transport, and usually is very dense to maximize cell cultivation in a given space. In one embodiment, the reactor includes a wall forming an interior with a packed or fixed bed comprised of a packing material (such as fibers, beads, spheres, or the like) for promoting the adhesion and growth of cells. The material is located in a compartment within the interior of the reactor, which compartment may comprise an upper portion of a hollow, vertically extending tube. A second compartment is provided within the interior of the reactor for conveying fluid to and from the material of compartment at least partially forming the fixed bed. Typically, the packing material should be arranged to maximize the surface area for cell growth, with 1,000 square meters being considered an advantageous amount of surface area (which, for example, may be achieved using medical grade polyester microfibers as the packing material). In one embodiment, evenly-distributed media circulation may be achieved by a built-in magnetic drive impeller, ensuring low shear stress and high cell viability. The cell culture medium flows through the fixed-bed from the bottom to the top. At the top, the medium falls as a thin film down the outer wall where it takes up O₂ to maintain high KLa. in the bioreactor. This waterfall oxygenation, together with a gentle agitation and biomass immobilization, enables the bioreactor to achieve and maintain high-cell densities.

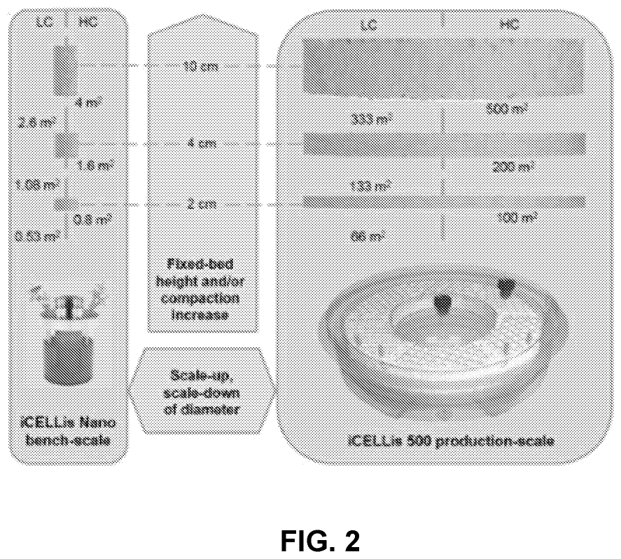

As used herein, the “bed height” is a parameter of either the bioreactor or the so-called fixed bed of the bioreactor (where the bioreactor uses a fixed bed). In many commercial adherent bioreactor systems, the bed of macrocarriers is provided with the bioreactor in a single-use (disposable) system and therefore the bed height of the bioreactor and the bed height of the fixed bed are generally synonymous. Bed heights of 2 cm, 4 cm, or 10 cm are illustrated in FIG. 2.

In some embodiments, the bioreactor has a bed height in the range of about 1 cm to about 15 cm, or in the range of about 2 cm to about 12 cm. In some embodiments, the bioreactor has a bed height of about 2 cm, 3 cm, 4 cm, 5 cm, 6 cm, 7 cm, 8 cm, 9 cm, 8 cm, 9 cm, 10 cm, 11 cm, or 12 cm. In some embodiments, the bioreactor has a bed height of about 2 cm. In other embodiments, the bioreactor has a bed height of about 10 cm. In certain embodiments, the bioreactor has a reactor volume of 500 ml to 1500 ml, 500 ml to 100 ml, about 500 ml, about 600 ml, about 700 ml, about 800 ml, about 900 ml, about 1,000 ml, about 1,100 ml, about 1,200 ml, or about 1,500 ml.

In some embodiments, the fixed bed has a bed height in the range of about 1 cm to about 15 cm, or in the range of about 2 cm to about 12 cm. In some embodiments, the fixed bed has a bed height of about 2 cm, 3 cm, 4 cm, 5 cm, 6 cm, 7 cm, 8 cm, 9 cm, 8 cm, 9 cm, 10 cm, 11 cm, or 12 cm. In some embodiments, the fixed bed has a bed height of about 2 cm. In other embodiments, the fixed bed has a bed height of about 10 cm. In certain embodiments, the fixed bed has a reactor volume of 500 ml to 1500 ml, 500 ml to 100 ml, about 500 ml, about 600 ml, about 700 ml, about 800 ml, about 900 ml, about 1,000 ml, about 1,100 ml, about 1,200 ml, or about 1,500 ml.

Exemplary parameters for iCELLis® bioreactors are provided in Table A. FIG. 2 illustrates the relationships between bed height, diameter, volume, and compaction.

TABLE A
Configurations of iCELLis ® bioreactors at small and manufacturing scale

| | Bioreactor | Fixed-Bed | | | Surface Area | |
|---|---|---|---|---|---|---|
| | Diameter | Height | Fixed-Bed | Bioreactor | (m²) Low | High |
| Bioreactor | (mm) | (mm) | Volume (L) | Volume (L) | Compaction | Compaction |
| iCELLis | 110 | 20 | 0.04 | 1 | 0.53 | 0.8 |
| Nano | | | | | | |
| iCELLis | 110 | 40 | 0.08 | 1 | 1.06 | 1.6 |
| Nano | | | | | | |
| iCELLis | 110 | 100 | 0.2 | 1 | 2.65 | 4 |
| Nano | | | | | | |
| iCELLis | 860 | 20 | 5 | 70 | 66 | 100 |
| 500/100 | | | | | | |
| iCELLis | 860 | 40 | 10 | 70 | 133 | 200 |
| 500/500 | | | | | | |
| iCELLis | 860 | 100 | 25 | 70 | 333 | 500 |
| 500/500 | | | | | | |

Source: https://biotech.pall.com/

In some embodiments, the fixed bed contains a macrocarrier (e.g., a matrix). In some embodiments, the macrocarrier is a fiber matrix. In some embodiments, the macrocarrier is a carbon fiber matrix. The macrocarrier may be selected from woven or non-woven microfibers, polyester microfibers (e.g., medical-grade polyester microfibers) porous carbon and matrices of chitosans. The microfibers may optionally be made of PET or any other polymer or biopolymer. In some embodiments, the macrocarriers include beads. The polymers may be treated to be compatible with cell culture, if such treatment is necessary. Suitable low-compaction macrocarriers that may be used include the proprietary macrocarriers provided with the iCELLis® bioreactor system; however other suitable low-compaction macrocarriers known in the art or prospectively developed can be substituted.

Suitable macrocarrier, matrix or “carrying material” are mineral carriers such as silicates, calcium phosphate, organic compounds such porous carbon, natural products such as chitosan, polymers or biopolymers compatible with cells growth. The matrix can have the form of beads with regular or irregular structure, or may comprising woven or non-woven microfibers of a polymer or any other material compatible with cell growth. The packing can also be provided as a single piece with pores and or channels. The packing in the recipients can have a variety of forms and dimensions. In some embodiments the matrix is a particulate material of solid or porous spheres, flakes, polygons. Typically a sufficient amount of matrix is used to avoid movement of the matrix particles within the recipient upon use, as this may damage cells and may have an influence on the circulation of gas and/or medium. Alternatively the matrix consists of an element which fits into the inner recipient or into a compartment of the recipient, and having an adequate porosity and surface. An example hereof is a carbon matrix (Carboscale) manufactured by Cinvention (Germany). In some embodiments, the fiber matrix has a surface area accessible to the cell of between about 150 cm²/cm³ and about 1000 cm²/cm³. In some embodiment, the bed height of the low-compaction macrocarriers is 2 cm, 4 cm, or 10 cm.

In some embodiments, the adherent bioreactor is an iCELLis® bioreactor having a modular fixed bed. Manufacturing of viral vectors in the iCELLis® bioreactor is described, for example, in WO2018007873A1 and US20180195048A1, which are incorporated herein by reference in its entirety. Commercially available bioreactors such as the iCELLis® Nano and 500/100 bioreactors Bioreactors (Pall® Life Sciences, Port Washington, N.Y.) may include a bioreactor system with a removable, disposable, or single use fixed bed that provides a large growth surface area in a compact bioreactor volume. Compared to a standard stirred-tank bioreactor using microcarriers, such systems avoid several delicate and time-consuming procedures, including manual operations, sterilization and hydration of microcarriers and bead-to-bead transfers from preculture to final process. As described herein, such bioreactors may enable process at a large scale (e.g., 500 square meters) culture area equivalent and harvest fluid volumes of up to 1500 to 2000 L, which is advantageous for industrial scale production of virus (e.g., for use in lentivirus production). As exemplified herein, such devices may enable further advantages such as low cell inoculums; reaching of optimal cell density for infection at a short preculture period; and/or optimization of MOI, media and serum concentrations during the culture growth phase. Such devices may be configured to allow rapid perfusion of the cells in culture, e.g., such that 90% or more of the cells experience the same medium environment. Moreover, and without wishing to be bound to theory, a single-use or disposable fixed bed may allow streamlined downstream processing to maximize the productivity as well as reduce the foot print of the process area even with scale up equivalent to several large scale conventional culture vessels. As such, advantageous productivity and purity may be achieved with minimal steps and costs.

In some embodiment, the predetermined cell density achieved prior to the transfecting step is 1-10,000×10³ cells per cm². In some embodiment, the predetermined cell density achieved prior to the transfecting step is 1-1,000×10³ cells per cm², 1,000-2,000×10³ cells per cm², 2,000-3,000×10³ cells per cm², 3,000-4,000×10³ cells per cm², 4,000-5,000×10³ cells per cm², 5,000-6,000×10³ cells per cm², or 6,000-7,000×10³ cells per cm². In some embodiment, the predetermined cell density achieved prior to the transfecting step is 1-100×10³ cells per cm², 100-200×10³ cells per cm², 200-300×10³ cells per cm², 300-400×10³ cells per cm², 400-500×10³ cells per cm², 500-600×10³ cells per cm², or 600-700×10³ cells per cm². In some embodiment, the predetermined cell density achieved prior to the transfecting step is 150-300×10³ cells per cm². In some embodiment, the predetermined cell density achieved prior to the transfecting step is 150-200×10³ cells per cm², 200-250×10³ cells per cm², or 250-300×10³ cells per cm².

Producer Cells and Cell Culture

The producer cells may be any producer cell or cell line suitable for production of a lentiviral vector and adapted, or adaptable, for growth in adherent mode. In an embodiment, the producer cells are HEK293 cells or a derivative thereof, optionally HEK293T cells or a derivative thereof. In an embodiment, the producer cells are adherent HEK293 or HEK293T cells.

Producer cells, e.g., HEK293 or HEK293T cells, may be cultured in a variety of cell culture media, such as Dulbecco's Modified Eagle's Medium (DMEM), Minimum Essential Media (MEM), Iscove's Modified Dulbecco's Medium (IMDM), OptiPRO™, EX-CELL® 293, or Pro293™ media. Culture conditions for producer cells, including the above cell types, are known and described in a variety of publications, or alternatively culture medium, supplements, and conditions may be purchased commercially, such as for example, as described in the catalog and additional literature of Cambrex Bioproducts (East Rutherford, N.J.). In certain embodiments, the producer cells are cultured in serum-free media. Known serum-free media that may be used include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolyzate (Gibco and other manufacturers) are also adequately known in the prior art. The media additives based on plant hydrolyzates have the special advantage that contamination with viruses, mycoplasma or unknown infectious agents can be ruled out.

In some embodiments, the producer cells comprises one or more polynucleotides that facilitate viral replication (e.g., polynucleotides encoding Gag-pol, Rev, and/or env genes). In some embodiments, the producer cells are derived from a packaging cell line. In some embodiment, the producer cells are not derived from a packaging cell line. In an embodiment, the cell line is engineered to express one or more of Gag-pol, rev, and Env(VSVG) without helper plasmids. Appropriate Gag-pol, Rev, and Env polypeptides, plasmids encoding these, and packaging cell lines that express these polypeptides are known and available in the art.

Transfection Reagents

While in certain embodiments, the transfection reagent is calcium phosphate, in some embodiments, other transfection reagents are used. Suitable transfection reagents can be, e.g., PEIPro™ (PolyPlus), JetPEI™, linear PEI or any polyethylene imine derivative, or any other functionally-equivalent transfection reagent. In an embodiment, the transfection reagent is a cationic polymer, e.g., Lentifectin™. In embodiments, the transfection reagent mixture comprises any buffer suitable for use in cell culture, including but not limited to phosphase, citrate-phosphate, or 4-(2-hydroxyethyl)-1-piperazineethanesulfonic acid (HEPES) buffer. In one embodiment, the buffer is HEPES, and the mixture is prepared in HEPES-buffered saline (e.g., UltraSALINE A). In some embodiments, the buffer is any buffer suitable for cell culture known in the art and compatible with calcium phosphate transfection reagent. In certain embodiments, the buffer is buffered saline, e.g., HEPES-buffered saline, such as UltraSALINE A). In certain embodiments, the buffer is ((N,N-bis[2-hydroxyethyl]-2-aminoethanesulfonic acid) (BES). In embodiments, the transfection reagent mixture has a neutral pH, e.g. pH 6.5, 6.6, 6.7, 6.8, 6.9, 7.0, 7.1, 7.2, 7.3, 7.4, 7.5, 7.6, 7.7, or 7.8, or in the range of pH 6.5-7.8. In certain embodiments, the transfection reagent mixture is at a pH of about 7.2 or about 7.4. In one embodiment, the transfection reagent mixture is at a pH of about 7.2.

In an embodiment, the transfection reagent mixture comprises about 90-200 mM CaPho and has a pH of about 7.0-7.6 at 37° C. In an embodiment, the transfection reagent mixture comprises about 110 mM CaPho and has a pH of about 7.2 at 37° C. In an embodiment, the transfection reagent mixture comprises about 120 mM CaPho and has a pH of about 7.2 at 37° C. In an embodiment, the transfection reagent mixture comprises about 125 mM CaPho and has a pH of about 7.2 at 37° C. In an embodiment, the transfection reagent mixture comprises about 130 mM CaPho and has a pH of about 7.2 at 37° C. In an embodiment, the transfection reagent mixture comprises about 140 mM CaPho and has a pH of about 7.2 at 37° C. In an embodiment, the transfection reagent mixture comprises about 150 mM CaPho and has a pH of about 7.2 at 37° C. In an embodiment, the transfection reagent mixture comprises about 160 mM CaPho and has a pH of about 7.2 at 37° C. In an embodiment, the transfection reagent mixture comprises about 170 mM CaPho and has a pH of about 7.2 at 37° C. In an embodiment, the transfection reagent mixture comprises about 180 mM CaPho and has a pH of about 7.2 at 37° C.

In an embodiment, the transfection reagent mixture comprises about 110 mM CaPho and has a pH of about 7.4 at 37° C. In an embodiment, the transfection reagent mixture comprises about 120 mM CaPho and has a pH of about 7.4 at 37° C. In an embodiment, the transfection reagent mixture comprises about 125 mM CaPho and has a pH of about 7.4 at 37° C. In an embodiment, the transfection reagent mixture comprises about 130 mM CaPho and has a pH of about 7.4 at 37° C. In an embodiment, the transfection reagent mixture comprises about 140 mM CaPho and has a pH of about 7.4 at 37° C. In an embodiment, the transfection reagent mixture comprises about 150 mM CaPho and has a pH of about 7.4 at 37° C. In an embodiment, the transfection reagent mixture comprises about 160 mM CaPho and has a pH of about 7.4 at 37° C. In an embodiment, the transfection reagent mixture comprises about 170 mM CaPho and has a pH of about 7.4 at 37° C. In an embodiment, the transfection reagent mixture comprises about 180 mM CaPho and has a pH of about 7.4 at 37° C.

In an embodiment, the transfection reagent mixture comprises about 1 to about 150 μg/mL of the one or more DNA polynucleotides. In an embodiment, the transfection reagent mixture comprises about 1 to about 120 μg/mL of the one or more DNA polynucleotides. In an embodiment, the transfection reagent mixture comprises about 1, 5, 10, 15, 20, 25, 30, 35, 40, 45, 50, 55, 60, 65, 70, 75, 80, 85, or 90 μg/mL of the one or more DNA polynucleotides. In an embodiment, the transfection reagent mixture comprises about 10, 20, or 30 μg/mL of the one or more DNA polynucleotides. In an embodiment, the transfection reagent mixture comprises about 20 μg/mL of the one or more DNA polynucleotides. In some embodiments, the transfection reagent mixture is applied twice or at least twice, so that, e.g., 2×20 μg/mL of the one or more DNA polynucleotides is delivered to the cells.

In various embodiments, the process conditions are selected from the enumerate examples provided in Table B. Other combinations of conditions are possible. For example, in some embodiments, the pH is 7.2 or 7.4. In some embodiments, the calcium phosphate concentration is 125 mM or 180 mM. In some embodiments, the DNA concentration in the transfection reagent mixture is 20 μg/mL or 20 μg/mL. In some embodiments, transfection reagent mixture is added one time or two times.

TABLE B
Non-Limiting Examples of Run Conditions

| | | | | Txn | Seed | Trx | Reactor | Total |
|---|---|---|---|---|---|---|---|---|
| | | | DNA | Mix | (1,000 | Volume | Volume | volume |
| Ex. | pH | Ca | (ug/mL) | Temp | cells/cm2) | (mL) | (mL) | (mL) |
| 1 | 7.2 | 125 | 20 | RT | 2 | 600 | 800 | 2,400 |
| 2 | 7.4 | 180 | 20 | 37 | 2 | 1,000 | 800 | 2,400 |
| 3 | 7.4 | 180 | 20 | 37 | 2 | 600 | 1,000 | 3,000 |
| 4 | 7.4 | 180 | 20 | 37 | 2 | 600 | 800 | 6,300 |
| 5 | 7.4 | 180 | 20 | 37 | 2 | 600 | 800 | 5,800 |
| 6 | 7.4 | 180 | 20 | 37 | 2 | 600 | 800 | 5,800 |
| 7 | 7.4 | 180 | 20 | 37 | 2 | 600 | 800 | 1,800 |
| | | | (x2) | | | | | |
| 8 | 7.4 | 180 | 20 | 37 | 2 | 600 | 800 | 1,800 |
| 9 | 7.4 | 180 | 20 | 37 | 1.5 | 600 | 800 | 4,050 |
| 10 | 7.4 | 180 | 20 | 37 | 1.5 | 600 | 800 | 3,450 |
| | | | (x2) | | | | | |
| 11 | 7.4 | 180 | 20 | 37 | 1.5 | 600 | 800 | 1,400 |
| 12 | 7.2 | 125 | 20 | 37 | 1.5 | 200 | 800 | 3,400 |
| 13 | 7.4 | 180 | 20 | 37 | 1.5 | 200 | 800 | 3,400 |
| 14 | 7.4 | 180 | 40 | 37 | 1.5 | 400 | 800 | 3,400 |

In certain embodiments, the methods of the disclosure result in an increase in cell count of at least about 5%, at least about 10%, at least about 15%, at least about 20%, or at least about 25% compared to a reference method. In certain embodiments, the methods of the disclosure result in an increase in titer of at least about 5%, at least about 10%, at least about 15%, at least about 20%, or at least about 25% compared to a reference method. In certain embodiments, the methods of the disclosure result in an increase in transducing units per liter of at least about 5%, at least about 10%, at least about 15%, at least about 20%, or at least about 25% compared to a reference method. In certain embodiments, the methods of the disclosure result in an increase in vector copies number per liter of at least about 5%, at least about 10%, at least about 15%, at least about 20%, or at least about 25% compared to a reference method. In certain embodiments, the methods of the disclosure result in an increase in vector genomes per liter of at least about 5%, at least about 10%, at least about 15%, at least about 20%, or at least about 25% compared to a reference method. In certain embodiments, the methods of the disclosure result in an increase in infectious titer of at least about 5%, at least about 10%, at least about 15%, at least about 20%, or at least about 25%. In some embodiments, the reference method comprises transfection with a transfection reagent other than CaPho. In some embodiments, the reference method comprises transfection with a transfection reagent mixture comprising CaPho at about 80 mM, about 100 mM, about 150 mM, about 180 mM or about 200 mM. In some embodiments, the reference method comprises transfection with a transfection reagent mixture having a pH of about 6.4, about 6.6, about 6.8, about 7.0, about 7.4, about 7.6, or about 7.8.

In certain embodiments, HEK293 cells are cultured in an iCELLis® bioreactor in DMEM cell culture medium at 37° C. in a volume of about 800 mL. A transfection reagent mixture comprising DMEM cell culture media with UltraSALINE A at pH 7.2 with 125 mM CaPho and with about 20 μg/mL of Chim.CD18-LV vector plasmid in about total 200 mL volume is added to the bioreactor. About 6 hours later, continuous harvesting of media begins and continues gradually over about 60 hours. After harvest, harvested material is stored for further purification.

Polynucleotides

In various embodiments, the producer cells are transfected or transduced with one or more polynucleotides. In some embodiments, they are transfected or transduced with a polynucleotide comprising an expression cassette, e.g., an expression cassette encoding a polypeptide or polynucleotide of interest, such as a therapeutic polypeptide or a therapeutic RNA. In particular embodiments, they are transfected with one or more polynucleotides encoding viral proteins necessary or desired for generating viral vectors, e.g., lentiviral vectors.

In an embodiment, the polynucleotide comprises a lentiviral vector gene expression cassette comprising a gene of interest or encoding a polypeptide of interest, optionally selected from the group consisting of R-type-specific pyruvate kinase (RPK), integrin subunit beta 2 (ITGB2), Fanconi Anemia complementation group A (FANCA), Fanconi Anemia complementation group C (FANCC), Fanconi Anemia complementation group G (FANCG), T Cell Immune Regulator 1 (TCIRG1), chloride voltage-gated channel 7 (CLCN7), tumor necrosis factor ligand superfamily member 11 (TNFSF11), Pleckstrin Homology And RUN Domain Containing M1 (PLEKHM1), TNF receptor superfamily member 11a (TNFRSF11A) and Osteoclastogenesis Associated Transmembrane Protein 1 (OSTM1), or a functional fragment or variant thereof.

In some embodiments, the producer cells comprise one or more polynucleotides that facilitate viral replication (e.g., polynucleotides encoding Gag-pol, Rev, and/or env genes). In some embodiments, the producer cells are derived from a packaging cell line. In an embodiment, the producer cell line is engineered to express one or more of Gag-pol, rev, and Env(VSVG) without helper plasmids. Thus, in certain embodiments, the producer cells are only transfected with the plasmid comprising the expression cassette comprising a gene of interest or encoding a polypeptide of interest.

In some embodiments, gene(s) encoding viral structural proteins are provided to the producer cells on the same or on a different polynucleotide from a gene expression cassette comprising a gene of interest or encoding a polypeptide of interest. For examples, the cells may be transfected with 1, 2, 3, or 4 plasmids expressing Gag-pol, rev, and/or Env(VSVG). In particular embodiments, the cells are transfected with four plasmids, wherein one plasmid encodes Gag-pol, one plasmid encodes Rev, one plasmid encodes Env, and one plasmid comprises the gene expression cassette comprising a gene of interest or encoding a polypeptide of interest, e.g., a therapeutic polypeptide Appropriate Gag-pol, Rev, and Env polypeptides, plasmids encoding these, and packaging cell lines that express these polypeptides are known and available in the art.

### Lentiviral Vectors, Pharmaceutical Compositions and Uses Thereof

In some embodiments, the disclosure provides a recombinant lentiviral vector produced by any method of the disclosure.

In some embodiments, the disclosure provides a pharmaceutical composition comprising the recombinant lentiviral vector produced by any method of the disclosure and a pharmaceutically acceptable carrier, diluent or excipient.

In some embodiments, the disclosure provides a pharmaceutical composition comprising a lentiviral vector or vector particle, wherein the vector particle is at a concentration of between 1×10⁷ and 1×10⁹ vector particles (vp) per mL. In some embodiments, the pharmaceutical composition comprises vector particle at a concentration of at least about 1×10⁷, at least about 1×10⁷, at least about 1×10⁸, at least about 1×10⁹, at least about 1×10¹⁰ or at least about 1×10¹¹ vector particles (vp) per mL. In some embodiments, the pharmaceutical composition comprises vector particle at a concentration of at least 1×10⁷, at least 1×10⁷, at least 1×10⁸, at least 1×10⁹, at least 1×10¹⁰ or at least 1×10¹¹ vector particles (vp) per mL. In some embodiments, the pharmaceutical composition comprises vector particle at a concentration of about 1×10⁷, about 1×10⁷, about 1×10⁸, about 1×10⁹, about 1×10¹⁰ or about 1×10¹¹ vector particles (vp) per mL. In certain embodiments, the recombinant lentiviral vector was produced by any method of the disclosure.

In certain embodiments, the recombinant lentiviral vector was produced by any method of the disclosure.

In particular embodiments, the disclosure provides a pharmaceutical composition comprising a population of cells, wherein a plurality of the cells is transduced by a recombinant lentiviral vector. In some embodiments, the population of cells was contacted with or infected with the recombinant lentiviral vector produced by any method of the disclosure.

The present invention includes pharmaceutical compositions and formulations comprising vectors as described herein and a pharmaceutically-acceptable carrier, diluent or excipient. The vectors can be combined with pharmaceutically-acceptable carriers, diluents and reagents useful in preparing a formulation that is generally safe, non-toxic, and desirable, and includes excipients that are acceptable for primate use. Examples of such excipients, carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, dimethyl sulfoxide (DMSO), fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the formulations are sterile.

In some embodiments, the methods are performed in accordance with Current Good Manufacturing Practices. Manufactured in accordance with Current Good Manufacturing Practices means that the formulation prepared for administration is sufficiently safe to permit administration to a human subject under controlling regulations and government authorizations. Generally, the controlling regulations and authorizations will dictate that the formulation meet pre-approved acceptance criteria regarding identity, strength, quality and purity. Acceptance criteria include numerical limits, ranges, or other suitable measures of test results used to determine whether a formulation meets the Current Good Manufacturing Practices. A specification sets forth the analytical procedures that are used to test conformance with the acceptance criteria. Formulations can be assessed in batches. A batch is a specific quantity of a formulation tested to ensure compliance with acceptance criteria.

The compositions or formulations can be included in a container, pack, or dispenser, e.g. syringe, e.g. a prefilled syringe, together with instructions for administration.

Where necessary or beneficial, compositions and formulations can include a local anesthetic such as lidocaine to ease pain at a site of injection.

In some embodiments, the pharmaceutical compositions and formulations provided herein comprise a therapeutically effective amount of viral vectors as disclosed herein in a mixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the tumor suppressor gene contained in the adenoviral vector delivery system, may be in the range of 6.5 to 7.75, preferably 7 to 7.5, and most preferably 7.2 to 7.4.

The lentiviral vectors and pharmaceutical composition produced may be used directly or stored.

The lentiviral vectors and pharmaceutical compositions may be used to treat or prevent a disease or disorder in a subject in need thereof, e.g., by administering them to a subject, or indirectly, e.g., by infecting a cell with the lentiviral vector, and then administering the cell to the subject as a therapeutic cell. In particular embodiments, the cell was obtained from the subject before being infected with the lentiviral vector.

In some embodiments, the disclosure provides for use of the recombinant lentiviral vector produced by any method of the disclosure or pharmaceutical composition of the disclosure to provide a polypeptide encoded by the polynucleotide to a cell.

In some embodiments, the disclosure provides for use of the recombinant lentiviral vector produced by the any method of the disclosure or pharmaceutical composition of the disclosure to treat a disease or disorder in a mammalian subject in need thereof. In some embodiments, the disease or disorder is Pyruvate Kinase Deficiency, Leukocyte Adhesion Deficiency, Fanconi Anemia, and/or osteopetrosis.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed.

The disclosure is further described in the following Examples, which do not limit the scope of the disclosure described in the claims

### EXAMPLES

### Example 1

### Optimized Manufacturing of Lentiviral Vector in Icellis Reactor

The following example demonstrates optimization of parameters used for production of lentiviral vectors in the iCELLis® system. Test parameters are provided in Table 1.

TABLE 1
Run Parameters

| | | | | | | Txn | Seed | Trx | Reactor | Total |
|---|---|---|---|---|---|---|---|---|---|---|
| | Bio- | | | | DNA | Mix | (1,000 | Volume | Volume | volume |
| Run | reactor | Type | pH | Ca | (ug/mL) | Temp | cells/cm2) | (mL) | (mL) | (mL) |
| 1 | 1 | 0.53 | 7.2 | 125 | 20 | RT | 2 | 600 | 800 | 2,400 |
| | 2 | 0.53 | 7.4 | 180 | 20 | 37 | 2 | 1,000 | 800 | 2,400 |
| | 3 | 0.53 | 7.4 | 180 | 20 | 37 | 2 | 600 | 1,000 | 3,000 |
| | 4 | 4 | 7.4 | 180 | 20 | 37 | 2 | 600 | 800 | 6,300 |
| 2 | 5 | 4 | 7.4 | 180 | 20 | 37 | 2 | 600 | 800 | 5,800 |
| | 6 | 4 | 7.4 | 180 | 20 | 37 | 2 | 600 | 800 | 5,800 |
| | 7 | 0.8 | 7.4 | 180 | 20 | 37 | 2 | 600 | 800 | 1,800 |
| | | | | | (x2) | | | | | |
| | 8 | 0.8 | 7.4 | 180 | 20 | 37 | 2 | 600 | 800 | 1,800 |
| 3 | 9 | 2.6 | 7.4 | 180 | 20 | 37 | 1.5 | 600 | 800 | 4,050 |
| | 10 | 2.6 | 7.4 | 180 | 20 | 37 | 1.5 | 600 | 800 | 3,450 |
| | | | | | (x2) | | | | | |
| | 11 | 0.53 | 7.4 | 180 | 20 | 37 | 1.5 | 600 | 800 | 1,400 |
| | 12 | | | | | | | | | |
| 4 | 13 | 2.6 | 7.2 | 125 | 20 | 37 | 1.5 | 200 | 800 | 3,400 |
| | 14 | 2.6 | 7.4 | 180 | 20 | 37 | 1.5 | 200 | 800 | 3,400 |
| | 15 | 2.6 | 7.4 | 180 | 40 | 37 | 1.5 | 400 | 800 | 3,400 |
| | 16 | | | | | | | | | |

For Runs 1-3, the plasmid used to generate a lentiviral vector to test the system encodes an enhanced Green Fluorescent Protein (EGFP) transgene. For Run 4, the plasmid used was the Chim.CD18-LV vector, which encodes a CD18 transgene.

In each run, four bioreactors were used. They were configured with 0.53, 0.8, 2.6, or 4 type, such that the surface area of the macrocarriers was 0.53, 0.8, 2.6, or 4 square meters in the iCELLis Nano. Cells were seeded at 1,500 or 2,000 cells/cm² and grown until the cell density reached 150-200,000 cells/cm², at which point the transfection reagent mixture was added over 6 hours with continuous mixing at 1 cm/s. Cells counts at transfection and end of run are provided in Table 2.

TABLE 2
Cell Counts at Transfection and End of Run

| | Bio- | Tranfection | End Run | | |
|---|---|---|---|---|---|
| Run | reactor | (cells/cm²) | (cells/cm²) | GFP % | MFI |
| 1 | 1 | 150-200,000 | 375,000 | >95% | ~8,500 |
| | 2 | 150-200,000 | 375,000 | >95% | ~6,000 |
| | 3 | 150-200,000 | 375,000 | >95% | ~6,000 |
| | 4 | 150-200,000 | 10,000-60,000 | 67-90 | ~2,000 |
| 2 | 5 | 216,726 | 363,309- | 79.5-87.1 | 600 |
| | | | 509,892 | | |
| | 6 | 360,611 | 430,755- | 60-69.8 | 1,200 |
| | | | 503,597 | | |
| | 7 | 268,884 | 329,136 | 69.6 | 1,800 |
| | 8 | 260,000 | 310,251 | 91.4 | 5,400 |
| 3 | 9 | 270,683 | 526,672 | 77.7 | 2,750 |
| | 10 | 199,640 | 375,887 | 73 | 2,230 |
| | 11 | 152,877 | 366,906 | 98.9 | 6,800 |
| | 12 | N/A | N/A | N/A | N/A |
| 4 | 13 | ND | ND | N/A | N/A |
| | 14 | ND | ND | N/A | N/A |
| | 15 | ND | ND | N/A | N/A |
| | 16 | N/A | N/A | N/A | N/A |

ND = not determined.
N/A = not applicable.

As shown in Table 1, the transfection reagent mixture contained either 20 or 40 μg/mL of DNA with either 125 or 180 mM calcium phosphate at pH 7.2 or 7.4. Where indicated by “2×” twice the volume of transfection reagent mixture was used. The transfection reagent and DNA was mixed at either room temperature or at 37° C. The transfection reagent mixture was made in DMEM cell culture media with UltraSALINE A (a HEPES-based saline solution) used to buffer the solution. 200, 400, 600, or 1,000 mL of transfection reagent mixture was added to the reaction up to a total volume of 800 or 1,000 mL.

After 6 hours, harvesting of the media was begun. At this point the bioreactor was emptied and 800 ml of prewarmed media was added to the bioreactor. The perfusion media (5 L) was connected and perfusion started. Harvesting was performed gradually over 60 hours, such that in total 4-5 reactor volumes (roughly 2,400-6,300 mL) were added. After harvest, harvested material was stored at room temperature (RT) or at 4° C.

As summarized in Tables 2 (above) and Tables 3-6, the collected material was assessed for total cell count; percentage of GFP+ cells (GFP %) and mean fluorescence intensity (MFI) (when EGFP vector was used); physical titer based on reverse transcriptase quantitative polymerase chain reaction (RT-qPCR) measured in vector genomes (vg); viral particles (vp) based on p24 level; transducing units (TU); and vector copy number (VCN). The p24 level was measured by enzyme-linked immuneabsorbance assay using the Lenti-X™ p25 Rapid Titer Kit from Takara®. The titer of lentiviral particles was determined by assuming there are approximately 2000 molecules of p24 per Lentiviral Particle (LP); therefore, 1 LP contains 2,000×24×10³/(6×10²³) g of p24=8×10-5 pg of p24; or 1 ng p24=1.25×107 LPs.

Run 13 resulted in the highest yield of the 16 runs performed. Harvested material was stored at either room temperature or 4° C.

TABLE 3
Physical Titer by RTqPCR (VG)

| | | | | | | Total vg | vg/cm2 |
|---|---|---|---|---|---|---|---|
| Run | Bioreactor | 24 hr | 48 hr | 60 hr | Sum | (RTqPCR) | (vg basis) |
| 1 | 1 | 8.92E+08 | 2.37E+09 | 3.03E+09 | 6.29E+09 | 6.29E+09 | 1.19E+06 |
| | 2 | 4.80E+08 | 6.23E+09 | 2.24E+09 | 8.95E+09 | 8.95E+09 | 1.69E+06 |
| | 3 | 5.09E+08 | 3.71E+09 | 6.58E+09 | 1.08E+10 | 1.08E+10 | 2.04E+06 |
| | 4 | 1.89E+09 | 9.07E+09 | 1.78E+10 | 1.78E+10 | 1.78E+10 | 4.45E+05 |
| 2 | 5 | ND | ND | ND | ND | ND | N/A |
| | 6 | ND | ND | ND | ND | ND | N/A |
| | 7 | ND | ND | ND | ND | ND | N/A |
| | 8 | ND | ND | ND | ND | ND | N/A |
| 3 | 9 | ND | ND | ND | ND | 8.08E+09 | 3.11E+05 |
| | 10 | ND | ND | ND | ND | 8.59E+09 | 3.30E+05 |
| | 11 | ND | ND | ND | ND | 9.36E+09 | 1.77E+06 |
| | 12 | N/A | N/A | N/A | N/A | N/A | N/A |
| 4 | 13 | 6.45E+09 | 1.08E+10 | 8.16E+09 | 2.54E+10 | 2.54E+10 | 9.77E+05 |
| | 14 | 4.28E+08 | 2.66E+09 | 2.38E+09 | 5.47E+09 | 5.47E+09 | 2.10E+05 |
| | 15 | 2.32E+08 | 5.68E+09 | 4.80E+09 | 1.07E+10 | 1.07E+10 | 4.12E+05 |
| | 16 | N/A | N/A | N/A | N/A | N/A | N/A |

TABLE 4
Physical Particle titer by p24 ELISA (VP)

| | | | | | | Total vp | vp/mL | vp/cm2 |
|---|---|---|---|---|---|---|---|---|
| | | | | | | in | (p24 | (p24 |
| Run | Bioreactor | 24 hr | 48 hr | 60 hr | Sum | harvest | basis) basis) | |
| 1 | 1 | ND | ND | ND | ND | ND | N/A | N/A |
| | 2 | ND | ND | ND | ND | ND | N/A | N/A |
| | 3 | ND | ND | ND | ND | ND | N/A | N/A |
| | 4 | ND | ND | ND | ND | ND | N/A | N/A |
| 2 | 5 | ND | ND | ND | ND | ND | N/A | N/A |
| | 6 | ND | ND | ND | ND | ND | N/A | N/A |
| | 7 | ND | ND | ND | ND | ND | N/A | N/A |
| | 8 | ND | ND | ND | ND | ND | N/A | N/A |
| 3 | 9 | ND | ND | ND | ND | 9.45E+11 | 2.33E+08 | 3.63E+07 |
| | 10 | ND | ND | ND | ND | 2.88E+11 | 8.35E+07 | 1.11E+07 |
| | 11 | ND | ND | ND | ND | 3.26E+11 | 2.33E+08 | 6.15E+07 |
| | 12 | N/A | N/A | N/A | N/A | N/A | N/A | N/A |
| 4 | 13 | 4.4E+11 | 1.8E+12 | 1.16E+12 | 3.40E+12 | 3.40E+12 | 1.00E+09 | 1.31E+08 |
| | 14 | 4.92E+10 | 3.11E+11 | 2.09E+11 | 5.7E+11 | 5.7E+11 | 1.68E+08 | 2.19E+07 |
| | 15 | 2.24E+10 | 5.04E+11 | 3.76E+11 | 9.03E+11 | 9.03E+11 | 2.66E+08 | 3.47E+07 |
| | 16 | N/A | N/A | N/A | N/A | N/A | N/A | N/A |

TABLE 5
Infectious Titer—Transducing Unit titer by GFP (TU)

| | | | | | | Total TU | | |
|---|---|---|---|---|---|---|---|---|
| | | 24 hr | 48 hr | 60 hr | Harvest | in | | |
| Run | Bioreactor | TU/ml | TU/ml | TU/ml | TU/ml | harvest | TU/cm² | vg/TU |
| 1 | 1 | 4.81E+05 | 1.26E+06 | 5.72E+05 | 2.31E+06 | 1.85E+09 | 3.79E+05 | 3.40 |
| | 2 | 2.42E+05 | 1.09E+06 | 4.63E+05 | 1.80E+06 | 1.44E+09 | 2.71E+05 | 6.22 |
| | 3 | 2.48E+05 | 8.90E+05 | 3.37E+05 | 1.48E+06 | 1.41E+09 | 2.67E+05 | 7.66 |
| | 4 | 5.42E+05 | 5.40E+05 | 3.35E+05 | 4.87E+05 | 2.95E+09 | 7.37E+04 | 6.04 |
| 2 | 5 | 3.69E+04 | 6.73E+04 | ND | 4.22E+04 | 7.66E+08 | 1.92E+04 | N/A |
| | 6 | 5.41E+04 | 1.00E+05 | ND | 7.39E+04 | 1.23E+09 | 3.08E+04 | N/A |
| | 7 | 4.62E+04 | 1.56E+05 | ND | 4.93E+04 | 4.14E+08 | 5.18E+04 | N/A |
| | 8 | 8.97E+04 | 3.90E+05 | ND | 1.50E+05 | 5.69E+08 | 7.11E+04 | N/A |
| 3 | 9 | ND | ND | ND | 7.46E+05 | 3.02E+09 | 1.16E+05 | 2.67 |
| | 10 | ND | ND | ND | 3.19E+05 | 1.10E+09 | 4.24E+04 | 7.8 |
| | 11 | ND | ND | ND | 7.53E+05 | 1.05E+09 | 1.99E+05 | 8.9 |
| | 12 | N/A | N/A | N/A | N/A | N/A | N/A | N/A |
| 4 | 13 | N/A | N/A | N/A | N/A | N/A | N/A | N/A |
| | 14 | N/A | N/A | N/A | N/A | N/A | N/A | N/A |
| | 15 | N/A | N/A | N/A | N/A | N/A | N/A | N/A |
| | 16 | N/A | N/A | N/A | N/A | N/A | N/A | N/A |

TABLE 6
Infectious Titer—Vector Copy Number (VCN) by qPCR

| Run | Bioreactor | VCN | VCN/cm2 | vp/NCN | vg/VCN | VCN/TU |
|---|---|---|---|---|---|---|
| 1 | 1 | 8.99E+09 | 1.70E+06 | N/A | 0.70 | 4.86 |
| | 2 | 4.76E+09 | 8.98E+05 | N/A | 1.88 | 3.31 |
| | 3 | 7.52E+09 | 1.42E+06 | N/A | 1.44 | 5.34 |
| | 4 | 9.22E+09 | 2.31E+05 | N/A | 1.93 | 3.13 |
| 2 | 5 | ND | ND | N/A | N/A | N/A |
| | 6 | ND | ND | N/A | N/A | N/A |
| | 7 | ND | ND | N/A | N/A | N/A |
| | 8 | ND | ND | N/A | N/A | N/A |
| 3 | 9 | 8.68E+09 | 3.34E+05 | 108.86 | 0.93 | 2.87 |
| | 10 | 3.36E+09 | 1.29E+05 | 85.74 | 2.56 | 3.05 |
| | 11 | 2.34E+09 | 4.42E+05 | 139.28 | 4.00 | 2.22 |
| | 12 | N/A | N/A | N/A | N/A | N/A |
| 4 | 13 | 2.51E+10 | 9.65E+05 | 135.46 | 1.01 | N/A |
| | 14 | 4.509E+09 | 1.73E+05 | 126.41 | 1.21 | N/A |
| | 15 | 9.303E+09 | 3.58E+05 | 97.07 | 1.15 | N/A |
| | 16 | N/A | N/A | N/A | N/A | N/A |

What is claimed:

1. A method of manufacturing a recombinant lentiviral vector, comprising:
a. culturing producer cells in culture media in an adherent mode on a matrix, wherein the matrix comprises low-compaction macrocarriers, in an adherent bioreactor having a bed height and a reactor volume until the producer cells achieve a predetermined cell density;
b. transfecting the producer cells with a transfection reagent mixture, wherein the transfection reagent mixture comprises one or more DNA polynucleotides, calcium phosphate (CaPho) at a neutral pH, and buffered saline (optionally, HEPES-buffered saline); and
c. harvesting the recombinant lentiviral vector, thereby generating harvested material.

2. The method of claim 1, wherein the adherent bioreactor is an iCELLis® bioreactor having a modular fixed bed.

3. The method of claim 1 or claim 2, wherein the bed height of the low-compaction macrocarriers is 2 cm, 4 cm, or 10 cm.

4. The method of any of claims 1-3, wherein the predetermined cell density achieved prior to the transfecting step is 150-300×10⁶ cells per cm².

5. The method of any of claims 1-4, wherein the transfection reagent mixture comprises about 125 mM CaPho and has a pH of about 7.2 at 37° C.

6. The method of any of claims 1-5, wherein the transfection reagent mixture comprises about 20 μg/mL of the one or more DNA polynucleotides.

7. The method of any of claims 1-6, wherein the transfecting step comprises adding to the adherent bioreactor about one volume of the transfection reagent mixture for each three volumes of culture media.

8. The method of any of claims 1-7, wherein the method comprises, after the transfecting step, recirculating the culture media through the matrix for about 5-7 hours while maintaining the pH at about 7.2.

9. The method of any of claims 1-8, wherein the harvesting step comprises maintaining the pH of the culture media at less than about pH 7.0.

10. The method of any of claims 1-9, wherein the harvesting step comprising perfusing the matrix with about 4 reactor volumes of harvesting media over about 24, 48, 60, or 72 hours.

11. The method of any of claims 1-10, wherein the method comprises processing the harvested material using a semi-closed or closed system, thereby generating purified material.

12. The method of claim 11, wherein the processing step comprises one or more of ion exchange chromatography and size exclusion chromatography.

13. The method of claim 11 or claim 12, wherein the processing step comprises concentrating the recombinant lentiviral vector by centrifugation of the harvested material in one or more centrifugal concentrators.

14. The method of claim 11 or claim 12, wherein the processing step comprises concentrating the recombinant lentiviral vector by tangential flow filtration.

15. The method of any one of claims 11-14, wherein the method comprises assaying the purified material to determine an infectious titer of the recombinant lentiviral vector.

16. The method of any one of claims 1-15, wherein the recombinant lentiviral vector manufactured by the method exhibits viral transduction efficacy that is increased by about 20% compared to recombinant lentiviral vector manufactured without optimization of process parameters.

17. The method of any one of claims 1-17, wherein the polynucleotide encodes a lentiviral vector gene expression cassette comprising a gene of interest or encoding a polypeptide of interest optionally selected from the group consisting of RPK, ITGB2, FANCA, FANCC, FANCG, TCIRG1, CLCN7, TNFSF11, PLEKHM1, TNFRSF11A and OSTM1.

18. The method of any of one claims 1-17, wherein lentiviral vector produced by the method is capable of achieving greater than 10% engraftment and repopulation of gene-modified cells when administered to a subject.

19. The method of any of one claims 1-18, wherein the lentiviral vector is a HIV-derived lentiviral vector.

20. The method of any one of claims 1-19, wherein the producer cells are HEK293 cells or a derivative thereof, optionally HEK293T cells or a derivative thereof.

21. A recombinant lentiviral vector produced by the method of any one of claims 1-20.

22. A pharmaceutical composition comprising the recombinant lentiviral vector of claim 21 and a pharmaceutically acceptable carrier, diluent or excipient.

23. A pharmaceutical composition comprising a population of cells, wherein a plurality of cells are transduced by a lentiviral vector.

24. Use of the recombinant lentiviral vector of claim 21 or pharmaceutical composition of claim 22 to provide a polypeptide encoded by the polynucleotide to a cell.

25. Use of the recombinant lentiviral vector of claim 21 or pharmaceutical composition of claim 22 to treat a disease or disorder in a mammalian subject in need thereof.

26. An adherent bioreactor adapted for use in the method of any one of claim 1-20.
